(19) United States
(12) Patent Application Publication
(10) Pub. No.: US 2023/0009747 A1
(43) Pub. Date: Jan. 12, 2023

(54) System And Method For Monitoring A Bodily Substance In A Human Orifice With A Wearable Device

(71) Applicant: Mark D. Noar, Owings Mills, MD (US)

(72) Inventor: Mark D. Noar, Owings Mills, MD (US)

(21) Appl. No.: 17/371,363

(22) Filed: Jul. 9, 2021

(51) Int. Cl. A61B 5/145; A61B 5/00

(52) U.S. Cl. CPC A61B 5/14507; A61B 5/6817; A61B 5/6819; A61B 5/682; A61B 5/0022; A61B 5/4839; A61B 5/4277; A61B 5/4294; A61B 5/746; A61B 5/6898; A61B 2560/0431

(57) ABSTRACT

A system and method is provided for monitoring a biological substance in a bodily orifice. The system includes a wearable device configured to be worn in a bodily orifice. A biosensor is carried by the wearable device and is constructed and arranged to obtain raw data regarding a biological substance in the orifice. The biosensor includes a processor circuit to provide processed data from the raw data, and a transmitter to wirelessly transmit the processed data to a second device.

### FIELD

The invention relates to a system and method for monitoring a bodily substance in an orifice such as the mouth, ears, nose, vagina and anus using a wearable device so as to provide information regarding the bodily substance indicative of a bodily disorder.

### BACKGROUND

An internal bodily disorder such as, gastroesophageal reflux disease (GERD), is typically diagnosed by an upper endoscopy whereby a flexible tube with a tiny camera is inserted into the esophagus to examine it and collect a sample of tissue (biopsy) if needed, or by esophageal pH monitoring whereby a monitor is inserted into the esophagus to learn if and when stomach acid enters it. These techniques are invasive, costly and require the patient to be treated in a hospital setting or at other medical facilities.

Accordingly, there is a need to provide a home-based system and method to non-invasively monitor a bodily substance in an orifice over time with a wearable device to provide information regarding a bodily disorder.

### SUMMARY

An object of the invention is to fulfill the need referred to above. In accordance with the principles of a present embodiment, this objective is obtained by a system for monitoring a biological substance in a bodily orifice. The system includes a wearable device configured to be worn in a bodily orifice. A biosensor is carried by the wearable device and is constructed and arranged to obtain raw data regarding a biological substance in the orifice. The biosensor includes a processor circuit to provide processed data from the raw data, and a transmitter to wirelessly transmit the processed data to a second device.

In accordance with another aspect of an embodiment, a method of monitoring a bodily substance in a body orifice places a wearable device with a biosensor in an orifice of a user so that a bio-receptor of the biosensor binds with a biological substance in the orifice. Raw data is obtained with the biosensor associated with the biological substance in the orifice over time. The raw data is processed in a microprocessor circuit to obtain processed data. The processed data is wirelessly transmitted to another device. Based on the processed data, the user is alerted to take therapeutic action.

Other objects, features and characteristics of the present invention, as well as the methods of operation and the functions of the related elements of the structure, the combination of parts and economics of manufacture will become more apparent upon consideration of the following detailed description and appended claims with reference to the accompanying drawings, all of which form a part of this specification.

### BRIEF DESCRIPTION OF THE DRAWINGS

The invention will be better understood from the following detailed description of the preferred embodiments thereof, taken in conjunction with the accompanying drawings, wherein like reference numerals refer to like parts, in which:

FIG. 1 is a block diagram of a system for monitoring a bodily substance in an orifice in accordance with a first embodiment of the invention.

FIG. 2 is a view a wearable device, in the form of a mouthguard, of the system.

FIG. 3 is a view of a wearable device, in the form of a vaginal or anal plug, of the system.

FIG. 4 is a view of a wearable device, in the form of an ear or nose plug, of the system.

FIG. 5 is a flow chart of method steps of an embodiment.

### DETAILED DESCRIPTION OF THE EXEMPLARY EMBODIMENTS

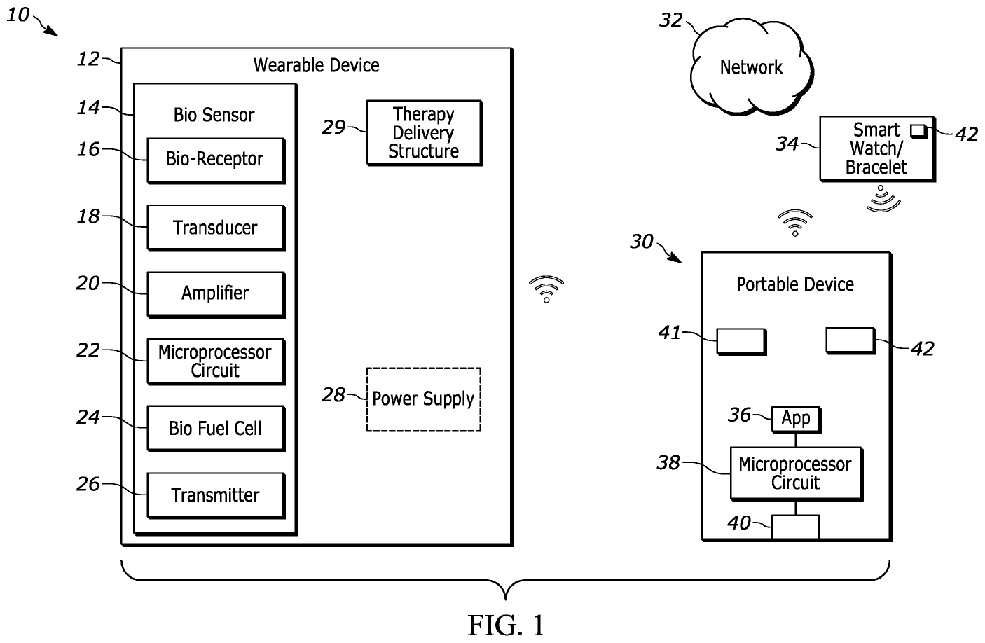

With reference to FIG. 1, an embodiment of a system for monitoring a bodily substance in an orifice is shown, generally indicated at 10. The system 10 includes a wearable device 12 constructed and arranged to be worn in a body orifice of a user. The user alone can insert the device 12 into and remove the device from the orifice. The system 10 also includes a biosensor 14 carried by the wearable device 12 for detecting and monitoring the bodily substance. The biosensor 14 preferably comprises a bio-marker or bio-receptor 16, preferably, but not limited to enzymes, proteins, cells, nucleic acids, or antibodies associated with the bodily substance; a transducer 18, preferably but not linted to electro-chemical electrodes, transistors, resistors, FET devices, and nano particle devices; an amplifier 20 to amplify an obtained raw electrical signal; a microprocessor circuit 22 configured to process the obtained raw electrical signal into a processed signal containing useful data; a bio-fuel cell to self-power the sensor 14; and a wireless transmitter 26 to transfer the data to a separate device. Alternatively, the transmitter can be separate from the biosensor 14, within the device 12, and a separate power supply 28 can be provided in the device 12 instead of the bio-fuel cell 24 to power the biosensor 14 and separate transmitter.

Biosensor 14 transforms physiological or biological information into electrical measurements using electronic circuits. Detected information is transmitted by transmitter 26 remotely to a data acquisition platform such a portable device 30 or a network server 32 using a wireless link for real-time monitoring and analysis. The portable device 30 (e.g., smart phone) can also communicate in a wireless manner with the network server 32. A smart watch or bracelet 34 can communicate in a wireless manner with the portable device 30. The server network 30 may include at least one of a telecommunication network such as a computer network (e.g., a LAN or a WAN), the Internet, cloud-based server, and a telephone network.

The portable device 30 can include an application (APP) 36 executed by a microprocessor circuit 38 that can analyze the processed data received from the biosensor 14. The data can be stored on the network 32, shared or retrieved via the network 32, or can be stored in a memory circuit 40 of the portable device 30. Also, the portable device 30 can receive data from the network 30 via receiver 41.

The biosensor 14 can employ electrochemical and electrical transduction mechanisms such as potentiometric, amperometric, field-effect transistor, resistive, capacitive, and inductive techniques. The electrical properties of the bio-sensors are converted into voltage, frequency, or phase using known interface or readout circuit techniques. Initially, bio-receptor 16 binds with biological substances of the user. The bio-receptor 16 send a response to the transducer 18 which generates an electrical or digital signal. The electrical signals are detected and amplified by amplifier 20 which sends the amplified signals to the microprocessor circuit 22. Processed signals are then sent by transmitter 26 to the portable device 30 or server network 32 for display or external review.

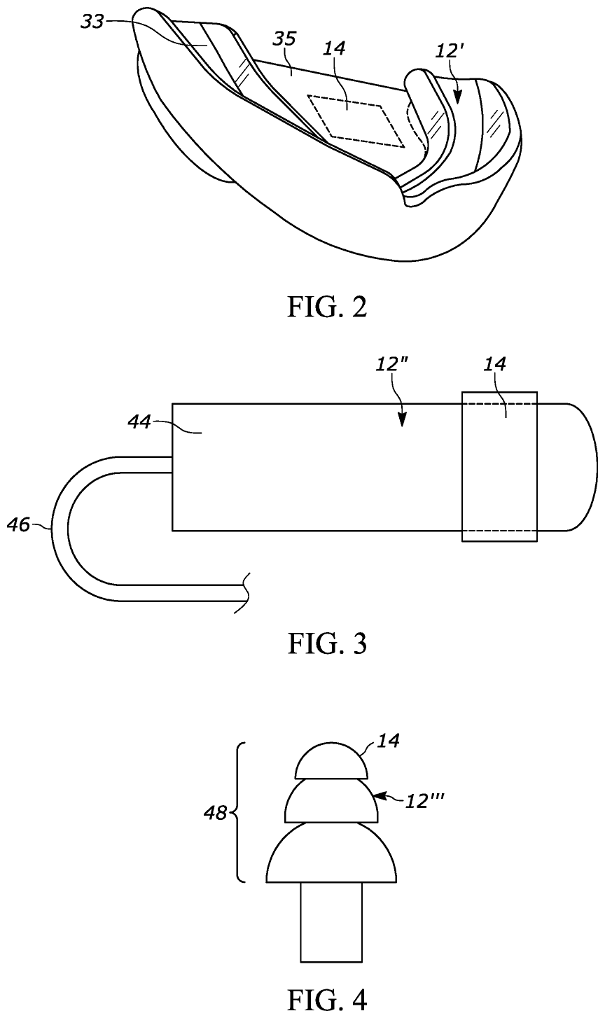

The biosensor 14 can be miniaturized down to one or more flexible integrated circuits to so that the wearable device is a point-of-care device. It is known that esophageal and extraesophageal gastric reflux (EEGR) and gastroesophageal reflux disease (GERD) can be diagnosed by detecting the presence of pepsin or pepsinogen of subjects suspected of having a reflux disorder or disease. This is typically done by employing single use lateral flow test strips. However, these strips cannot detect the pepsin or pepsinogen over time. Thus, with reference to FIG. 2, to detect a biological substance associated with GERD or EEGR over time, the wearable device can be in the form of a disposable mouthguard 12′ or retainer structure with the biosensor 14 incorporated therein so as to be able to detect the biological substance saliva (pepsin, pepsinogen), acid or pH levels in the user's saliva in the mouth, continuously over a time period or for a brief moment. The mouthguard 12′ can have a teeth receiving portion 33 and a pallet receiving portion 35 with the biosensor 14 preferably applied to the pallet receiving portion 35. When the user wears the device 12′ continuously over a time period, the user can enter data via the smart watch 34 or portable device 30 when the user experiences a reflux event, can record the start and end time of eating or drinking, can record the start and stop time of laying down, etc. Also, the smart watch 34 or portable device 30 can query the user when a reflex event is detected by the biosensor 14. For example, the smart watch 34 or portable device 30 can display, “A reflux event has occurred. Did you drink or eat recently?” The user can then respond, by using input 42, with an answer that is recorded with the event. The ability to obtain a quantitative value of the concentration of pepsin, pepsinogen, pH or acid in a saliva sample with the biosensor 14 enables one to match a disease or symptoms with such concentration. Although in the embodiment, the device 12′ is worn in the mouth (an orifice) and monitors saliva, the orifice can include passages connected there-with such as the tracheal and nasal passages and the device 12′ can sense biological substances associated with those connected passages.

With reference to FIG. 3, to detect a biological substance such as estrogen levels, the wearable device 12″ can be in the form of a disposable cylindrical plug 44, similar to tampon, but preferably of a disposable, semi-flexible and non-absorbent material. A string 46 is affixed to the plug for easy removal. Biosensor 14 is preferably wrapped around a portion of the periphery of the plug 44 so as to be able to detect estrogen in the user's vagina. Estrogen levels can provide important information about fertility, the health of a pregnancy, the menstrual cycle, and other health conditions. Estrogen is a hormone and levels thereof can be detected by detecting enzymes with the biosensor 14. The user can wear the device 12″ over time and can enter data via the smart watch 34 or portable device 30 when the user experiences an event relating to pregnancy or menstrual cycle, etc. Also, the smart watch 34 or portable device 30 can query the user when an event is detected by the biosensor 14. For example, the smart watch 34 or portable device 30 can display, “An event has occurred. Has menstrual cycle begun?”, or if the woman is trying to conceive, based on data from the biosensor 14, the smart watch 34 or portable device 30 can display, “Now is an optimal time to conceive” or if the woman is pregnant, the user can enter data, such as if blood spotting is occurring or other data.

The device 12″ can also be used to detect vaginal infections such as bacterial vaginosis by configuring the biosensor 14 to detect the biological substance such as the sialidase (SLD) enzyme or other enzymes, or yeast infections by configuring the biosensor 14 to detect the biological substance such as the Candida (yeast) antibody or other antibodies. If an infection is detected by the biosensor 14 , the transmitter 26 can transmit data to the portable device 30 or server network 32 and can alert the user to seek therapeutic action such as requesting an antibiotic from a doctor. The device 12″ can also be used to detect urinary incontinence by detecting urine. The device 12″ can also be inserted into the anus to detect, for example, proteins or antibodies associated with inflammatory bowel disease (IBD) in fluids (e.g. blood) in the rectum.

With reference to FIG. 4, to detect a biological substance associated with the ear or noise, the wearable device 12^{—} can be in the form of a disposable ear or nose plug having a portion 48 that is configured to be inserted into the ear or nasal orifice. The biosensor 14 is preferably couped to a periphery of the distal end of portion 48 so as to permit the biosensor 14 to obtain data from deep within the orifice. When used in the ear, the biosensor 14 detects a biological substance such as proteins in the ear fluid, puss or wax associated with inflammation or infection. When used in the nose, the biosensor 14 detects proteins (e.g., procalcitonin) associated with inflammation due to rhinosinusitis.

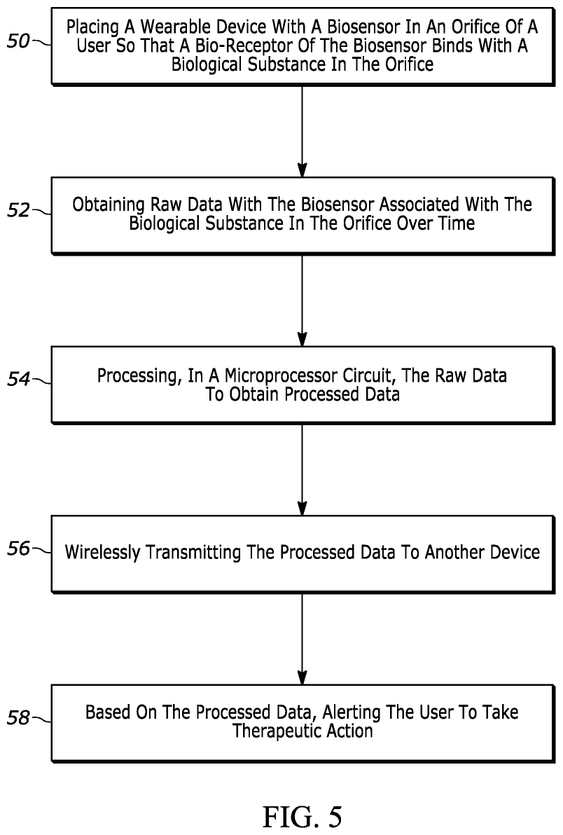

Thus, by employing the system 10 outlined above, with reference to FIG. 5, a method of for monitoring a biological substance in a bodily orifice in a user includes in step 50, placing the wearable device 12, 12″, or 12^{—} with biosensor 14 in an orifice of a user so that the bio-receptor 16 binds with the external (but within the cavity) biological substances of the user. In step 52, the biosensor 14 obtains raw data associated with the biological substance in the orifice over time. In step 54, the biosensor 14 processes, in microprocessor circuit 22, the raw data to obtain processed data. In step 56, the transmitter 26 of the biosensor wirelessly transmits the processed data to the portable device 30 or network server 32. In step 58, based on the processed data, the portable device 30 or network server 32 alerts the user to take therapeutic action. For example, the portable device 30 can alert the user to contact this/her doctor to order antibiotics when a viral infection is diagnosed. Alternatively, treatment can be performed, for example, by modulating the tissue with energy such as electro-magnetic frequency (EMF) or electric shock treatment from a therapy delivery structure 29 preferably included in the wearable device 12 (FIG. 1). For example if urinary incontinence is diagnosed by the biosensor 14, the therapy delivery structure 29 can deliver EMF or electric shock treatment to prevent or stop a bladder spasm. The therapy delivery structure 29 can be of the type disclosed in U.S. Patent Application Publication No. 20170332961 A1, the content of which is hereby incorporated by reference herein. The therapy delivery structure 29 can be separate from and outside of the device 12 (e.g., drugs or hormones) and implanted or disposed on the body of the patient and can communicated wirelessly with and be controlled by the network 32 or portable device 30. The microprocessor circuit 22 or 38 can signal the therapy delivery structure 29 to deliver the therapy. Thus, by use of the disposable, non-invasive and home useable system 10, the user can avoid the need to be diagnosed and even treated by a doctor at a doctor's office or medical facility.

The operations and algorithms described herein can be implemented as executable code within the microprocessor circuits 22, 38 as described, or stored on a standalone computer or machine readable non-transitory tangible storage medium that are completed based on execution of the code by a processor circuit implemented using one or more integrated circuits. Example implementations of the disclosed circuits include hardware logic that is implemented in a logic array such as a programmable logic array (PLA), a field programmable gate array (FPGA), or by mask programming of integrated circuits such as an application-specific integrated circuit (ASIC). Any of these circuits also can be implemented using a software-based executable resource that is executed by a corresponding internal processor circuit such as a micro-processor circuit and implemented using one or more integrated circuits, where execution of executable code stored in an internal memory circuit causes the integrated circuit(s) implementing the processor circuit to store application state variables in processor memory, creating an executable application resource (e.g., an application instance) that performs the operations of the circuit as described herein. Hence, use of the term “circuit” in this specification refers to both a hardware-based circuit implemented using one or more integrated circuits and that includes logic for performing the described operations, or a software-based circuit that includes a processor circuit (implemented using one or more integrated circuits), the processor circuit including a reserved portion of processor memory for storage of application state data and application variables that are modified by execution of the executable code by a processor circuit. The memory circuit 40 can be implemented, for example, using a non-volatile memory such as a programmable read only memory (PROM) or an EPROM, and/or a volatile memory such as a DRAM, etc.

The foregoing preferred embodiments have been shown and described for the purposes of illustrating the structural and functional principles of the present invention, as well as illustrating the methods of employing the preferred embodiments and are subject to change without departing from such principles. Therefore, this invention includes all modifications encompassed within the spirit of the following claims.

What is claimed is:

1. A system for monitoring a biological substance in a bodily orifice comprising:
a wearable device configured to be worn in a body orifice, and
a biosensor carried by the wearable device and constructed and arranged to obtain raw data regarding a biological substance in the orifice,
wherein the biosensor includes a processor circuit to provide processed data from the raw data, and a transmitter to wirelessly transmit the processed data to a second device.

2. The system of claim 1, wherein the wearable device is configured to be worn in a user's mouth and the biosensor is constructed and arranged to obtain the raw data from saliva as the biological substance.

3. The system of claim 2, wherein biosensor is constructed and arranged to detect levels of pepsin or pepsinogen in the saliva.

4. The system of claim 2, wherein biosensor is constructed and arranged to detect levels of acid or pH levels in the saliva.

5. The system of claim 2, wherein the wearable device is a disposable mouthguard having a teeth receiving portion and a pallet receiving portion, the biosensor being carried by the pallet receiving portion.

6. The system of claim 1, wherein the wearable device is configured to be worn in a user's vagina and the biosensor is constructed and arranged to obtain the raw data from estrogen as the biological substance.

7. The system of claim 1, wherein the wearable device is configured to be worn in a user's vagina and the biosensor is constructed and arranged to obtain the raw data from an enzyme or antibody associated with a vaginal infection as the biological substance.

8. The system of claim 6, wherein the wearable device is disposable cylindrical plug and the biosensor is wrapped around a portion of the periphery of the plug.

9. The system of claim 7, wherein the wearable device is disposable cylindrical plug and the biosensor is wrapped around a portion of the periphery of the plug.

10. The system of claim 1, wherein the wearable device is configured to be worn in a user's anus and the biosensor is constructed and arranged to obtain the raw data from the biological substance defined as proteins or antibodies in fluids in the rectum that are associated with inflammatory bowel disease.

11. The system of claim 10, wherein the wearable device is disposable cylindrical plug and the biosensor is wrapped around a portion of the periphery of the plug.

12. The system of claim 1, wherein the wearable device is configured to be worn in a user's ear and the biosensor is constructed and arranged to obtain the raw data from proteins in ear fluid, puss or wax associated with inflammation or infection.

13. The system of claim 1, wherein the wearable device is a disposable ear plug having a portion that is configured to be inserted into the ear orifice and the biosensor is carried on a periphery of the distal end of the portion.

14. The system of claim 1, wherein the wearable device is configured to be worn in a user's nose and the biosensor is constructed and arranged to obtain the raw data from proteins in ear nose associated with inflammation.

15. The system of claim 1, wherein the wearable device is a disposable nose plug having a portion that is configured to be inserted into the nose orifice and the biosensor is carried on a periphery of the distal end of the portion.

16. The system of claim 1, in combination with the second device, the second device being a portable smart phone.

17. The system of claim 16, further comprising therapy delivery structure constructed and arranged to be controlled by the smart phone, based on data from the biosensor, to deliver therapy to the user.

18. The system of claim 17, wherein the therapy delivery structure is constructed and arranged to deliver drugs, hormones, electromagnetic energy or shock treatment as the therapy.

19. A method of monitoring a bodily substance in a body orifice, the method comprising:
placing a wearable device with a biosensor in an orifice of a user so that a bio-receptor of the biosensor binds with a biological substance in the orifice,
obtaining raw data with the biosensor associated with the biological substance in the orifice over time,
processing, in a microprocessor circuit, the raw data to obtain processed data,
wirelessly transmitting the processed data to another device, and
based on the processed data, alerting the user to take therapeutic action.

20. The method of claim 19, wherein the wearable device is placed in the mouth and the biological substance is pepsin, pepsinogen, pH level or acid level in saliva.

21. The method of claim 19, wherein the wearable device is placed in a vagina and the biological substance is estrogen, an enzyme or antibody.

22. The method of claim 19 wherein the wearable device is placed in an ear and the biological substance is a protein in ear fluid, puss or wax.

23. The method of claim 19, wherein transmitting step transmits the processed data to a portable device and the step of alerting the user includes notifying the user via the portable device to take the therapeutic action.

24. The method of claim 23, wherein the therapeutic action includes controlling a therapy delivery structure to delivery therapy in the form of drugs, hormones, electromagnetic energy or shock treatment.
